(19) United States
(12) Patent Application Publication
(10) Pub. No.: US 2011/0060045 A1
(43) Pub. Date: Mar. 10, 2011

(54) ANTI-DIABETIC CATARACT COMPOUNDS AND THEIR USES

(75) Inventors: Yasuo Konishi, Kirkland (CA); Alaka Mullick, Montreal (CA)

(73) Assignee: NATIONAL RESEARCH COUNCIL OF CANADA, Ottawa, ON (CA)

(21) Appl. No.: 12/293,339

(22) PCT Filed: Mar. 23, 2007
(86) PCT No.: PCT/CA07/00477

§ 371 Date: Sep. 17, 2008

Related U.S. Application Data
(60) Provisional application No. 60/785,345, filed on Mar. 24, 2006.

(51) Int. Cl. A61K 31/195; A61K 31/192; A61P 27/12

(52) U.S. Cl. 514/561; 514/568

(57) ABSTRACT

The invention disclosed relates to the use of anti-glycation agents of formula (I),

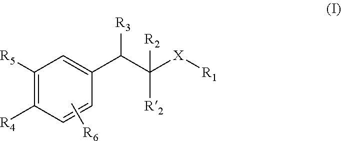

wherein
   X represents NR₇, wherein R₇ represents hydrogen atom or an acyl group derived from a linear, branched or cyclic C₁₋₁₀ aliphatic acid or a C₆₋₁₀ aromatic acid,
   R₁ represents hydrogen atom, NH₂, or a linear, branched or cyclic C₁₋₁₀ alkyl which may be substituted with a C₆₋₁₀ aromatic group,
   R₂ represents hydrogen atom, a linear, branched or cyclic C₁₋₁₀ alkyl, or COOH group,
   R′₂ represents hydrogen atom or a linear, branched or cyclic C₁₋₁₀ alkyl group,
   R₃ represents hydrogen atom, ═O, OR₈, SR₈, or NR₈R₉, wherein R₈ and R₉ represent hydrogen atom, a linear, branched or cyclic C₁₋₁₀ alkyl, or an acyl group derived from a linear or branched C₁₋₁₀ aliphatic acid or a C₆₋₁₀ aromatic acid, provided that R₈ and R₉ are not both an acyl group,
   R₄ and R₅ each independently represents OH, NH₂, or SH,
   R₆ represents hydrogen, F, Cl, Br, I, OR₁₀, or SR₁₀, wherein R₁₀ represents hydrogen or an acyl group derived from a linear or branched C₁₋₁₀ aliphatic acid or a C₆₋₁₀ aromatic acid, R₆ may be present more than once and each R₆ may be the same or different, a physiologically tolerated salt, prodrug, physiologically functional derivative or mixture thereof, such as (S)-isoproterenol, and its prodrug, (S)-isoproterenol dipivalate hydrochloride on the initiation of diabetic cataracts. (S)-Isoproterenol is a strong anti-glycation agent with an in vitro IC₅₀ value of 16.8±0.8 μM. (S)-isoproterenol dipivalate hydrochloride was prepared in eye drop form at 0.1% concentration and was applied to diabetic rats twice a day up to 30 weeks. No cataract was observed in non-diabetic rats with or without treatment of the prodrug. In diabetic rats without treatment of the prodrug (group III), 88% of eyes got cataract at 8.6±1.5 weeks. In diabetic rats with treatment of the prodrug, only 53% of the eyes initiated cataract at 8.6±1.2 weeks, and the remaining 26% of the eyes prolonged the initiation to 17.1±3.1 weeks. Furthermore, no cataract was observed in 21% of the eyes even at 30 weeks.

This application is a continuation in part of U.S. Ser. No. 10/492,553 filed on Oct. 15, 2002 which claims priority from U.S. Ser. No. 60/328,808 filed on Oct. 15, 2001 both of which are incorporated by reference in their entirety.

### FIELD OF THE INVENTION

This invention relates to uses of anti-diabetic compounds such as (S)-isoproterenol, especially (S)-isoproterenol dipivalate to prevent and delay the onset of diabetic cataracts. More specifically, this invention relates to a use of a prodrug form to deliver potent anti-glycation agents such as (S)-isoproterenol to the lens and a use of optically pure (S)-isoform as the adrenergically active (R)-isoproterenol may cause side effects.

### BACKGROUND OF INVENTION

More than 1 billion adults are overweight worldwide and at least 300 million of them are obese. Obesity and overweight pose a major risk for chronic diseases, including Type 2 diabetes, cardiovascular disease, hypertension, stroke and certain cancers. Obesity is increasing at an alarming rate worldwide, especially in developing countries. Diabetes, which is linked to obesity, is also increasing and causes a number of vascular complications in several organs in the form of retinopathy, nephropathy, neuropathy, hypertension, and peripheral ischemia. Diabetes also causes non-vascular complications such as cataract, glaucoma, arthropathy, periodontal diseases, and decreased skin elasticity.

Cataracts, which result from the opacification of the lens of the eye, are the leading cause of blindness worldwide. In fact, they account for approximately 42% of all blindness. Although diabetes is a major risk factor for cataract development, the probability of developing cataracts increases greatly with age even in the healthy, non-diabetic population. Approximately 50% of people between the ages of 65-75 and about 70% of people over the age of 75 have cataract. However, the present evidence indicates that cataracts reach maturity 10 years earlier in the diabetic population.

Diabetic cataract development involves multiple mechanisms. Three of them have been proven to contribute to cataract formation and therefore validated as targets for drug development (Stitt, 2001). They are pathways of glycation, oxidative stress and polyol. Glycation is non-enzymatic spontaneous chemical reactions between reducing sugars and amino groups of proteins, lipids, and nucleic acids. In diabetic cataract, glucose is the major source of reducing sugar and forms Schiff base, Amadori product and stable advanced glycation end products (AGEs) through a series of Maillard reaction. Some of the AGEs were identified including N^{ε}-carboxymethyllysine (CML), crossline, pentosidine, pyralline, Furoyl-furanyl imidazole, 1-alkyl-2-formyl-3,4-glycosyl-pyrrole, argpyrimidine, glyoxal lysine dimer (GOLD), deoxyglucosone-lysine dimer (DOLD), and methyl glyoxal lysine dimer (MOLD). Glycation also produces highly reactive α-dicarbonyl species, and induces oxidative stress, causing hyperglycemia-related diseases (Stitt, 2001). Hyperglycaemia produces intracellular oxidative stress. The resulting increase level of reactive oxygen species are signaling mediators damaging cellular targets through DNA oxidation, protein oxidation and lipid peroxidation. The oxidative stress also accelerates glycation. Thus, glycation and oxidative stress are somehow cross-linked. Another major mechanism is linked to increased flux through the polyol pathway, where aldose reductase is a rate-limiting enzyme in accumulation of sorbitol (Dagher et al., 2004).

Several potentially effective anti-cataract agents have been developed to block these pathways and have been investigated in animal, epidemiological and clinical studies (Kyselova et al., 2004). They could be classified as anti-oxidants, anti-glycation agents and aldose reductase inhibitors, although they are somehow related each other (Kyselova et al., 2004). Aldose reductase inhibitors have been developed to block polyol pathway. Flavonoids, benzopyrans, spirohydantoins, alkaloids, nonsteroidal anti-inflammatory agents, and quinones are structurally distinct but inhibit aldose reductase. Sorbinil, statil, tolrestat, alrestatin, epalrestat, and ALO1576 are some of the clinically studied inhibitors. However, none of them have proved clinically effective and, moreover, some have had deleterious side effects. Anti-oxidants reduce oxidative stress. In fact, the lens has endogenous antioxidants such as glutathione, vitamin C, vitamin E, carotenoids, superoxide dismutase, catalase, and Se-dependent GSH peroxidase. It should be noticed that some of the aldose reductase inhibitors are also anti-oxidants. Several anti-oxidants have been studied, e.g., α-lipoic acid is a potent antioxidant and reduces glucose level by increasing glucose uptake, resulting reduction in cataract formation (Packer et al., 2001). The third group of anti-cataract agents is anti-glycation agents. We screened anti-glycation activity of approximately 1,300 drugs or drug candidates. The most extensively studied anti-glycation agent is aminoguanidine, showing mixed results in animal tests. Pyridoxal-aminoguanidine, which is an anti-oxidant as well as anti-glycation agent, showed potent prevention of diabetic cataract in rat model. It is also reported that L-carnosine (prodrug: N-acetyl-L-carnosine) protects against the inactivation of esterase by glycation and thus ameliorates the pathological consequences of AGE formation (Yan & Harding, 2005), and improves the vision of cataract patients. In spite of these studies, no definitive drug to prevent or treat cataract has been approved by FDA.

We previously reported on the potent anti-glycation activity of catechols, dopamines and epinephrines (Yeboah et al., 2002). Drug repositioning of epinephrine has advantages for topical ocular applications, as dipivefrin, a prodrug of (R,S)-epinephrine, is a commercial eye drop drug to treat glaucoma. Thus, we developed eye drops to prevent/treat diabetes-related ocular complications such as cataract based on epinephrines.

One of the lead compounds is (S)-isoproterenol, which is considered as a safe agent for humans, in its prodrug format. (S)-isoproterenol d-bitartrate eye drop was administered to human eyes at a very high concentration of 10% and caused only brief mild conjunctival hyperemia and irritation. Topical administration of 20% (S)-isoproterenol HCl produced marked conjunctival hyperemia and mild miosis (a medical term for constriction of the pupil) that persisted for several hours (Kass et al., 1976). However, these concentrations are much higher than 0.1% of the prodrug preferably used in the current invention and it is unlikely that these adverse effects will be observed in human and animals. Also a large intravenous dose of (S)-isoproterenol appeared to have only slight and transient effects on blood pressure and pulse rate (Kass et al., 1976). The dipivaloyl prodrug of (R,S)-isoproterenol seems to be cardiovascularly inactive until it is activated to (R,S)-isoproterenol as the intravenous injection of the prodrug did not produce any unique cardiovascular effect that differentiates its action from those of (R,S)-isoproterenol in dog (Wang et al., 1977).

### SUMMARY OF THE INVENTION

The present invention provides a method for preventing and/or delaying the onset of diabetic cataracts. This involves applying to the eye of a patient in need of such a treatment.

The present invention utilizes a drug repositioning strategy to develop a novel application of epinephrines as anti-glycation agent. The present invention provides eye drop formulation for convenient topical ocular treatment of diabetes-related complications, more specifically diabetic cataract. The topical treatment reduces the amount of the dose and minimizes the potential side effects compared with systemic treatments.

In another embodiment of the invention, adrenergically inactive (S)-isomer (d-isomer) of epinephrines are used and the adrenergically active (R)-isoform (1-isoform) of adrenalines are excluded as they may reduce intraocular pressure, and may cause an increase of arterial blood pressure, tachycardia, local irritation, and mydriasis (Rowland and Potter, 1981).

In yet another embodiment of the invention, (S)-epinephrines are formulated into prodrug format in order to enhance the efficacy of the drug. The prodrug formulation is designed to increase the lipophilicity to effectively penetrate lipophilic cornea cell membranes.

In yet another embodiment of the invention, the prodrug is designed to be hydrolyzed at an appropriate rate by the enzyme(s) in cornea, aqueous humor and/or lens to deliver the drug to the lens.

In still yet another embodiment of the present invention, the prodrug is designed to penetrate a therapeutically effect concentration of the drug into the lens.

In still yet another embodiment of the present invention, the eye drop is designed to have a duration of several hours to avoid frequent inconvenient eye drop treatment.

The in vitro IC₅₀ is preferably less than 50 μM, especially less than about 40 μM, more especially less than about 30 μM.

The present invention provides a therapeutically effective dose to prevent/delay the onset of diabetic cataract. The concentration of compounds of the invention such as (S)-isoproterenol, the prodrug or the salt is preferably 0.01 to 10% w/v, especially preferably 0.01 to 5% w/v, particularly 0.01 to 1% w/v and especially about 0.1% w/v. The compound, the prodrug or the salt can be in unit dose form, for example in unit doses of 5-200 μL, more particularly 10-100 μL, especially 30-50 μL. 50 μL as the volume of each eye drop, i.e., 200 μL and 100 μL correspond to 4 and 2 eye drops each time. Some commercial eye drop product recommend the use of 2-3 eye drops each time. The prodrug contemplated is the dipivaloyl group though others, which have been reported in prodrug formulation such as diacetyl, dipropionyl, dibutyryl, dicyclopropanoyl, dibenzoyl, di(4-methylbenzoyl) groups may be used (Javinena and Jarvinenb, 1996). The salt commonly considered is the hydrochloride though other physiologically tolerated salts such as bitartrate, acetate or carbonate may be used. While animal studies have been carried out on rats it is reasonable to infer that this utility of compounds of the invention such as (S)-isoproterenol, the prodrug or the salt also applies to other mammals, including humans. With respect to purity of the optical isomers, preferably this is at least 97 or 98% w/w optical purity to reduce possible adrenergic and adverse effects of the (R)-isoform in long-term use.

### BRIEF DESCRIPTION OF THE FIGURES

FIG. 1. Blood glucose levels: The figure shows average blood glucose levels over the period of the experiment. Glucose levels were measured by weekly by tail vein puncture using a glucometer, for normal (filled diamond) or diabetic (filled squares) rats, receiving vehicle (A) or prodrug (B).

FIG. 2. Body weight: The figure shows average body weight over the period of the experiment. Normal (filled diamond) or diabetic (filled squares) rats receiving vehicle (A) or prodrug (B) were weighed weekly.

FIG. 3. Cataract progression: The figure shows photographs of cataract-bearing eyes that are representative of the 4 levels used to classify their severity. Level 0 is a normal eye (A). A faint pinkish hue characterizes a level 1 eye (B). A distinct white film in the eye that nevertheless still permits visualizing the pupil is defined as level 3 (C). The most severe form of cataract (level 4) covers the entire surface with a dense white film, precluding the detection of the pupil (D).

FIG. 4. Effect of (S)-isoproterenol on the initiation of cataract in diabetic rat eyes. The eyes of diabetic rats were topically treated with the prodrug (n=34) (filled circle) or with vehicle (n=26) (filled triangle) twice a day for up to 30 weeks. The percentage of non-cataractous lenses (level 0) is plotted over the 30-week period.

FIG. 5. Cytotoxicity of (S)-isoproterenol dipivalate in PC-12 cells. The cells were incubated for 5 min to 24 hr, followed with MTT assay to quantitate the survival of the cells.

FIG. 6. Drugs or drug candidates of which anti-glycation activity is known.

FIG. 7. Drugs or drug candidates of which anti-glycation activity has not been reported until discovered in our screening assay

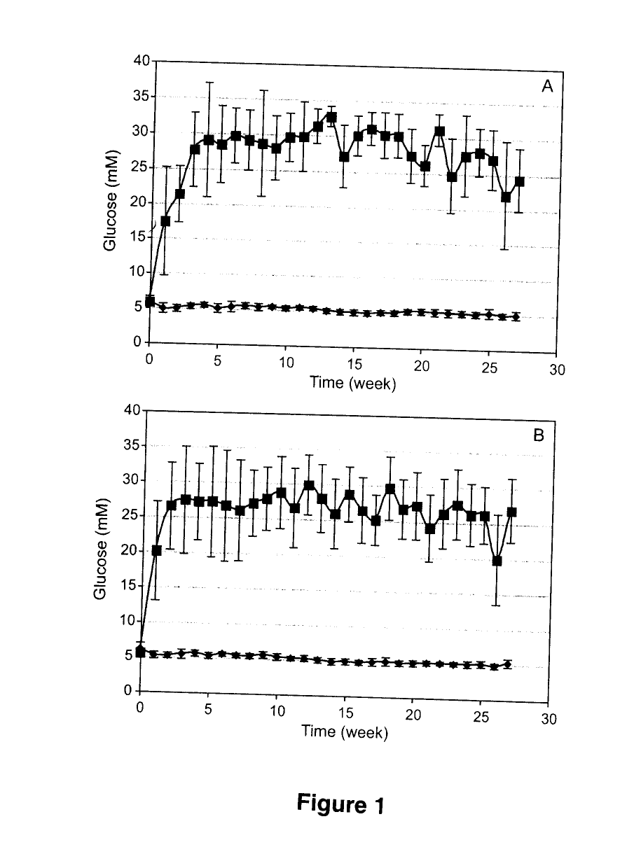

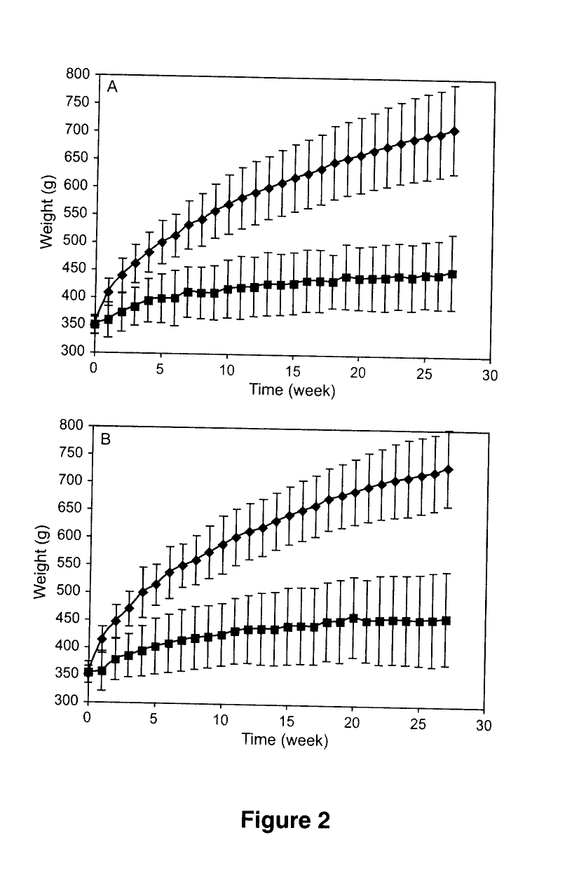

### DETAILED DESCRIPTION OF THE INVENTION

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems or devices, and any further applications of the principles of the invention as described herein, are contemplated as would normally occur to one skilled in the art to which the invention relates.

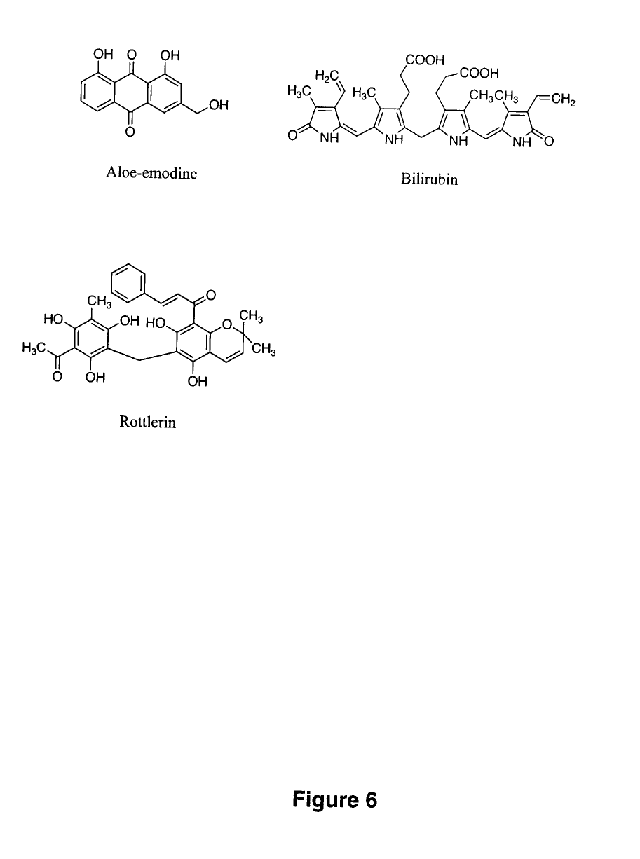

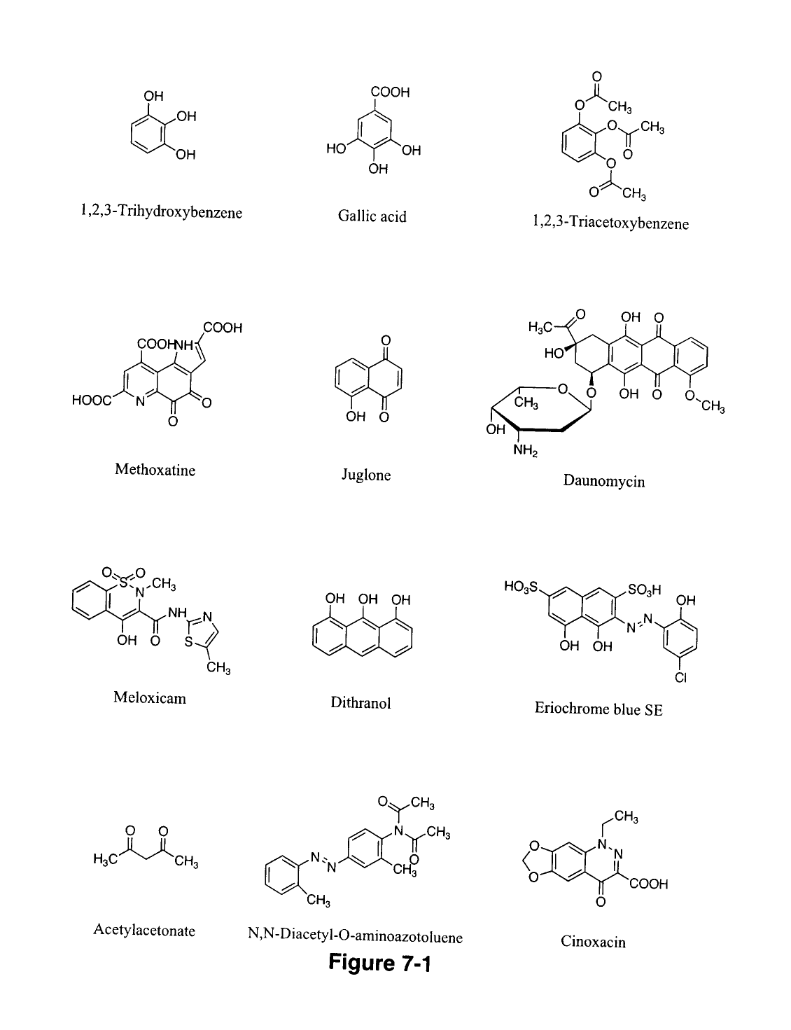

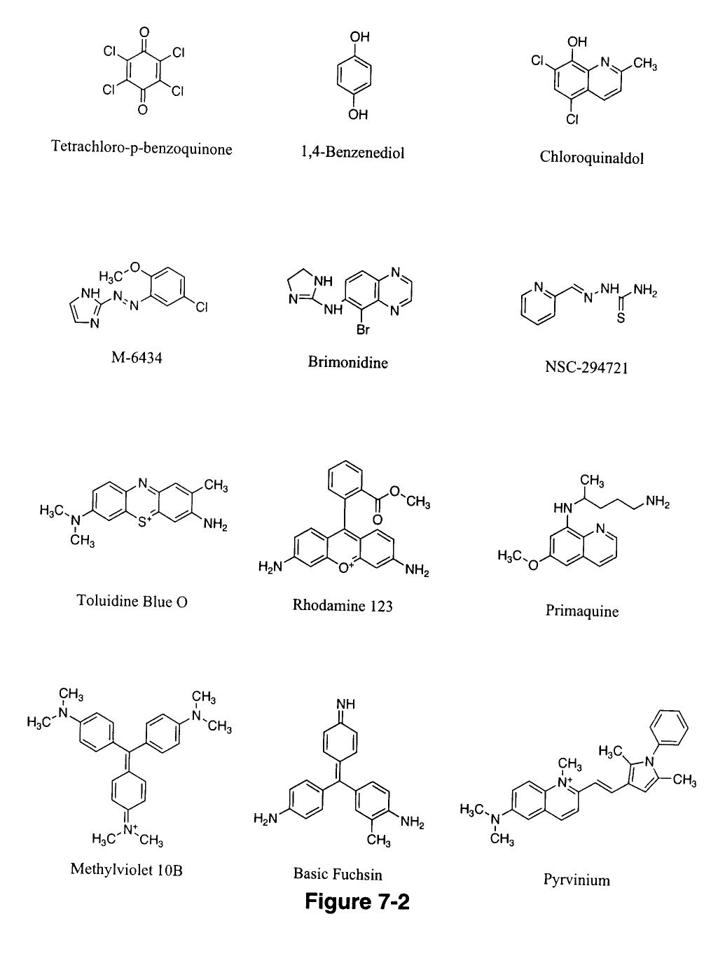

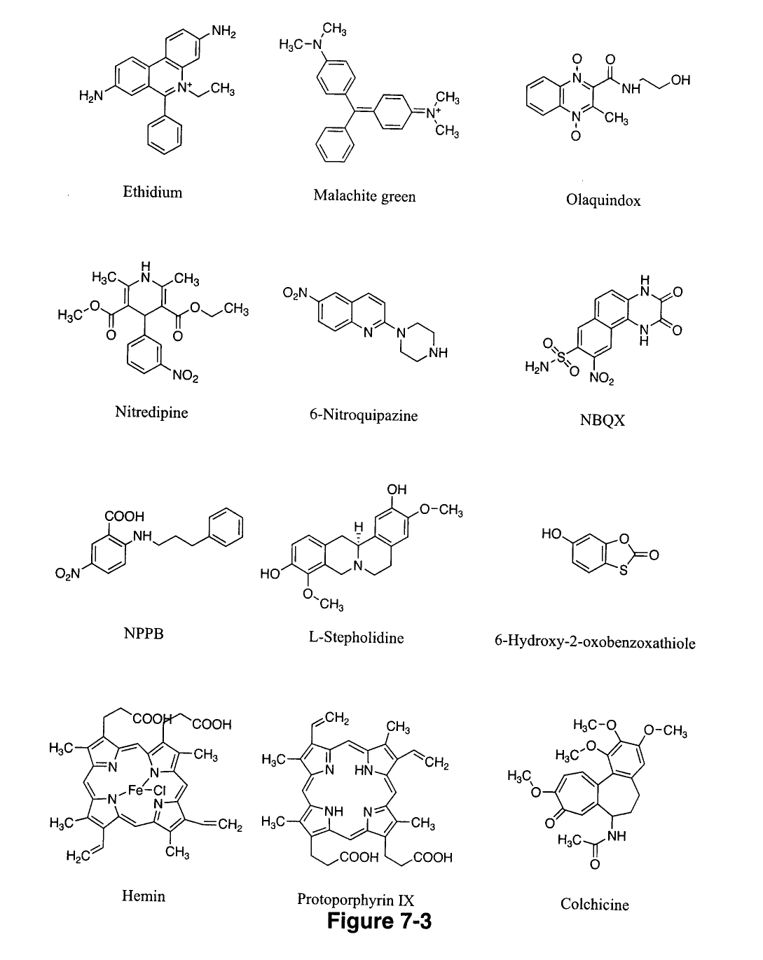

The present invention utilizes a drug repositioning strategy which is essentially the discovery of new use of existing drugs as anti-glycation agents. Some of the drugs discovered by using this strategy were listed by Yeboah et al. (2002). FIG. 7 shows other drugs of which IC₅₀ values were below 47 μg/mL. FIG. 6 illustrates some drugs or drug candidates for which anti-glycation activity is already known.

Among the drugs which showed anti-glycation activity, the present invention uses one of the most potent anti-glycation (S)-isoproterenol, of which IC₅₀ value was 16.8±0.8 μM, and its analogs. The catechol moiety of (S)-isoproterenol is essential for the anti-glycation activity based on their structure-activity relationship study.

The present invention provides novel application of (S)-isoproterenol (also known as d-isoproterenol) on prevention/delay of ocular complications of diabetic cataract formation. Since (S)-isoproterenol is a strong anti-glycation agent with an in vitro IC₅₀ value of 16.8+0.8 μM, it is likely that (S)-isoproterenol alleviates the effect of increased glycation in the lens, and therefore alleviates the symptoms of diabetic cataract.

The present invention excludes the use of adrenergically active (R)-isoproterenol (or l-isoproterenol) as (R)-isoproterenol reduces intraocular pressure as an adverse effect (Kass et al., 1976). (S)-isoproterenol bitartrate purchased from Aldrich (Oakville, Ontario, Canada) contained 2.0±0.3% of (R)-isoproterenol bitartrate impurity (see Experimental section).

The present invention uses (S)-isoproterenol dipivalate as a prodrug, which enhances and accelerates the ocular absorption and penetration through cornea. Dipivaloyl group also protects the 3,4-dihydroxyl group from chemical reactions such as oxidation during storage.

Among anti-glycation compounds of the present invention, of particular interest for prevention/delay of diabetic cataract are prodrugs of formula (II) of compounds of formula (I), which can be seen as analogs of (S)-isoproterenol dipivalate. (S)-Isoproterenol is clinically used in a racemic mixture of isoproterenol as sympathomimetic, bronchodilator, and anti-allergic drug. However, the active ingredient is (R)-isoproterenol and no therapeutic activity of (S)-isoproterenol has been reported with an exception of anti-glycation activity in our previous report (Yeboah et al., 2002).

The cytotoxicity of (S)-isoproterenol dipivalate prodrug was examined by using two cell lines. One is human corneal epithelial cells to which a high concentration of the prodrug (2.4 mM) is applied as eye drop. The other is PC12 cells which is a model cell of neuron with a concern that some of the prodrug may reach to neurons because the high lipophilicity of the prodrug may pass through blood brain barrier and blood retinal barrier. The PC12 cells were tolerant to up to 500 μM of the prodrug for the short time of incubation up to 2 hr (FIG. 5). The longer incubation up to 24 h was also carried out with a minimum media, resulting in the same tolerance of 500 μM (data not shown). The human cornea epithelial cells were much more tolerant to the prodrug as expected and no cytotoxicity was visibly noticed up to 25 mM of the prodrug under the microscope.

Streptozotocin was used to induce diabetes in rats. The blood glucose levels were monitored once a week over 27 week period for non-diabetic and diabetic rats. Control, Group I (receiving vehicle) and Group II (receiving prodrug) of non-diabetic animals (filled diamond) have a steady blood glucose level of 5.1±0.4 and 5.1±0.4 mM, respectively). An increase in blood glucose levels was noted for Group III (receiving vehicle) and Group IV (receiving prodrug) diabetic rats, during the first 2 weeks of diabetes induction. The glucose levels then stabilized at 28±4 and 27±5 mM, respectively. As the glucose level at around 5 mM is considered normal, rats in Group I and Group II are non-diabetic. Rats are considered diabetic when the blood glucose level exceeds 15 mM. Thus, all of the rats in Group III and Group IV are diabetic. The consistency of the blood glucose level between Groups I and II and between Groups III and IV shows that (S)-isoproterenol does not affect the blood glucose level and diabetes.

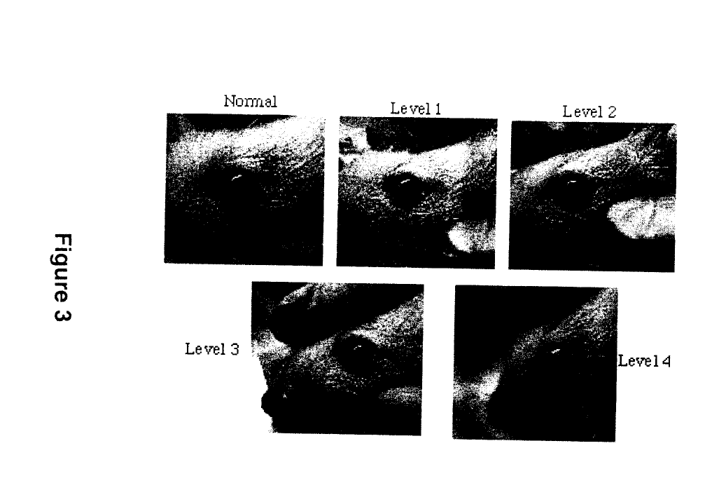

Another adverse effect of (S)-isoproterenol on body weight loss/gain was monitored. The weight gain during the experiments is essentially the same between Group I (receiving vehicle) and Group II (receiving prodrug), suggesting that no effect of (S)-isoproterenol in weight gain/loss of non-diabetic rats (FIG. 2). The weight gain is much less in the diabetic rats compared to those of non-diabetic rats (Chen et al., 2004); however, the weight gain of Group III (receiving vehicle) is essentially the same as that of Group IV (receiving prodrug), showing no effect of (S)-isoproterenol in weight gain/loss of diabetic rats (FIG. 2).

The initiation and progression of cataract in Streptozotocin-injected diabetic rats were monitored by scoring the severity of cataract in 4 levels, i.e., level 0 for a healthy eye, level 1 when a faint pinkish hue is discernable or the earliest stage of cataract is visually detected, level 2 when a white film is clearly detectable, level 3 when the film covers the entire eye, but the pupils are still visible, and level 4 when the pupil is not detected due to the formation of the white film. FIG. 3 allows the visual appreciation of cataract progression in the diabetic rats. During the experiments up to 30 weeks, no non-diabetic rats developed cataract even at the level 1. Thus, cataracts were not induced by age in the current invention.

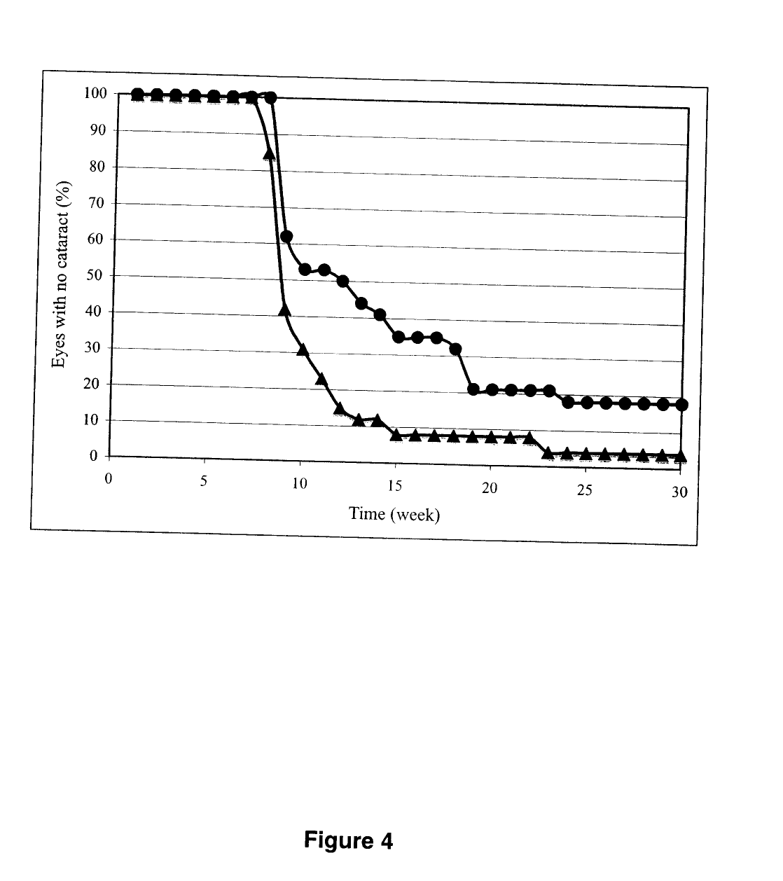

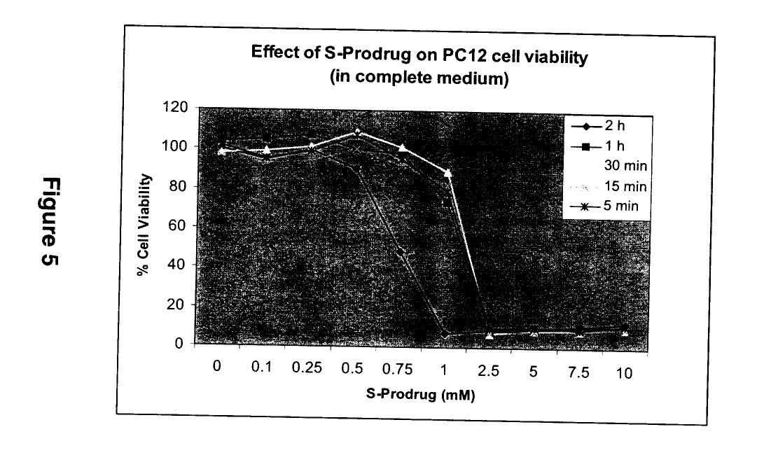

(S)-Isoproterenol delayed the initiation of cataract. On average, the diabetic rats with vehicle initiated cataract after 10.2±5.1 weeks, whereas the diabetic rats with the (S)-isoproterenol dipivalate initiated cataract after 15.0±8.3 weeks. Thus, (S)-isoproterenol delays cataract formation approximately 5 weeks (or 1.5-fold) in the diabetic rats. However, not all eyes initiate cataract at the same time, and their distribution shows a more drastic effect of (S)-isoproterenol (FIG. 4). In the diabetic control rats with the vehicle, 88% of the eyes initiated cataract at 8.6±1.5 weeks, and the remaining 3 eyes started cataracts at 14, 22 and >30 weeks, respectively. On the contrary, in the diabetic rats with the prodrug, 53, 26, 21% of the eyes initiated cataract at 8.6±1.2, 17.1±3.1, >30 weeks (except one eye was >23 weeks), respectively. In other words, (S)-isoproterenol delayed the initiation of cataract from 8.6 weeks to 17.1 weeks (double) for a quarter of the eyes, and furthermore, 21% of the eyes did not get cataract for more than 30 weeks (except one eye was >23 weeks). A delay of more than 30 weeks was unexpected because the polyol pathway, one of the major causes of cataract in rats, is not inhibited by anti-glycation agents. Since it is estimated that a delay in cataract formation of about 10 years would reduce the prevalence of visually disabling cataract by about 45%, our invention would have a significant social impact and reduce the number of required cataract surgery, which is a major drain on medical funds.

The progression of cataract was monitored by the time stayed at each level of cataract. Table 2 shows that the diabetic rats with vehicle (n=13), after initiating cataract, stayed for 1.18±0.76, 2.5±1.5, and 5.0±3.2 weeks at levels 1, 2, and 3, respectively, and then entered to the most severe level 4. Diabetic rats with the prodrug (n=17), after initiating cataract, stayed for 1.24±0.82, 1.88±1.09, and 5.4±1.9 weeks at levels 1, 2 and 3, respectively, and then entered to the most severe level 4. The differences of the time stayed at levels 1, and 2 and 3 between the diabetic rats with and without (S)-isoproterenol are within the experimental error. The polyol pathway plays significant role in rat cataract, where its progression is characteristically rapid compared to the slow progress in mouse and human eyes, in which polyol pathway plays minor role (Hegde et al., 2003). Thus, it is likely that (S)-isoproterenol has no effect on the polyol derived rapid progression of cataract; however, (S)-isoproterenol may delay the slow progression of cataract derived by glycation and/or oxidative stress in humans.

Adrenaline administration has been linked to increased cataract formation (Kyselova et al., 2004). Thus, it is unexpected that the homologous compound (S)-isoproterenol delayed cataract initiation. On the other hand, (S)-isoproterenol is one of the most potent anti-glycation agents (Yeboah et al., 2002) and could block or slow down glycation pathway of cataract formation. However, it is not known if (S)-isoproterenol has any effect on other potential mechanisms of cataract formation such as mitochondrial damage, calpain activation, cytoskeletal spectrin/fodrin proteolytic degradation, and fiber cell globulization (Hegde et al., 2003). Furthermore, the anti-cataract activity of (S)-isoproterenol could be directly or indirectly delay cataract initiation and nothing is known about the effect of (S)-isoproterenol on the progression of cataract when polyol pathway does not dominate the progression in such as human and mice. The detailed mechanism of (S)-isoproterenol-mediated inhibition of cataract initiation and progression is the subject of ongoing investigations.

The anti-glycation compounds according to the present invention represent a family of compounds in sharing a common core chemical structure. The compounds of the invention can be classified as anti-glycation agents. A low pKa (=8.72±0.05) of the aromatic dihydroxyl group of isoproterenol easily ionizes one of the aromatic OH, which may react with one of the □-dicarbonyl group of reactive intermediates such as 1-deoxyglucose, 3-deoxyglucosone, 2-glucosone, glucosone, methylglyoxal, and glyoxal. Another aromatic OH is then ionized and reacting with the remaining carbonyl, forming a six-member ring. If R₁ is hydrogen, dehydration may occur, followed by hydrolysis to from free carboxyl group and an aromatic OH. (S)-isoproterenol may also react with Amadori product and release Lys residue through rearrangement and hydrolysis. Similar reactions may occur with other glycation intermediates containing carbonyl group.

Glycation includes oxidative processes and is closely related to oxidative stress. In deed, several anti-inflammatory drugs, which have anti-oxidant activity, showed anti-glycation activity. Since the compounds of the invention may also inhibit glycation through the anti-oxidant activity, the anti-oxidant activity of (S)-isoproterenol and its prodrug (S)-isoproterenol dipivalate was measured against oxidative stress by H₂O₂ at the cellular level. The oxidative stress by H₂O₂ induces apoptosis in vitro and in vivo, and effective anti-oxidants such as N-acetyl-cysteine reduces the oxidative stress and protects the cells from the apoptosis. The anti-oxidant activities of (S)-isoproterenol and (S)-isoproterenol dipivalate were measured at the concentration range of 10-100 μM which is around its IC₅₀ value (16.8±0.8 μM). The incubation with H₂O₂ killed over 80% of the PC12 cells compared with the control without H₂O₂ treatment. Both (S)-isoproterenol and its prodrug did not show any protective effect, demonstrating that the anti-glycation activity of (S)-isoproterenol is not due to the anti-oxidant activity.

Compounds of formula (I)

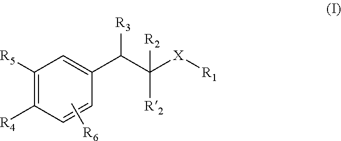

preferably compounds of formula (Ia)

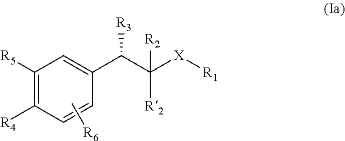

wherein:
X represents NR₇, wherein R₇ represents hydrogen atom or an acyl group derived from a linear, branched or cyclic aliphatic acid or an aromatic acid,
R₁ represents hydrogen atom, NH₂, or a linear, branched or cyclic C₁₋₁₀ alkyl which may be substituted with an aromatic group,

R₂ represents hydrogen atom, a linear, branched or cyclic C₁₋₁₀ alkyl, or COOH group,
R′₂ represents hydrogen atom or a linear, branched or cyclic C₁₋₁₀ alkyl group,

R₃ represents hydrogen atom, ═O, OR₈, SR₈, or NR₈R₉, wherein R₈ and R₉ represent hydrogen atom, a linear, branched or cyclic C₁₋₁₀ alkyl, or an acyl group derived from a linear or branched aliphatic acid or an aromatic acid, provided that R₈ and R₉ are not both an acyl group (only one of the chirality shown in the formula (I) is used unless R₃ itself inactivates the biological activity such as adrenergic activity),
R₄ and R₅ represent OH, NH₂, or SH,
R₆ represents hydrogen atom, halogen atom (F, Cl, Br or I), OR₁₀, or SR₁₀, wherein R₁₀ represents hydrogen atom or an acyl group derived from a linear or branched aliphatic acid or an aromatic acid. One or more of the same or different R₆ may substitute the aromatic ring.

Prodrugs of formula (II)

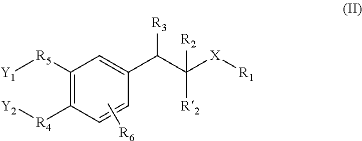

preferably compounds of formula (IIa)

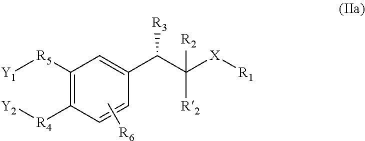

wherein:
X represents NR₇, wherein R₇ represents hydrogen atom or an acyl group derived from a linear, branched or cyclic aliphatic acid or an aromatic acid,
R₁ represents hydrogen atom, NH₂, or a linear, branched or cyclic C₁₋₁₀ alkyl which may be substituted with an aromatic group,
R₂ represents hydrogen atom, a linear, branched or cyclic C₁₋₁₀ alkyl, or COOH group,
R′₂ represents hydrogen atom or a linear, branched or cyclic C₁₋₁₀ alkyl group,
R₃ represents hydrogen atom, ═O, OR₈, SR₈, or NR₈R₉, wherein R₈ and R₉ represent hydrogen atom, a linear, branched or cyclic C₁₋₁₀ alkyl, or an acyl group derived from a linear, branched or cyclic aliphatic acid or an aromatic acid, provided that R₈ and R₉ are not both an acyl group,
R₄ and R₅ represent —O—, —NH— or —S—,
R₆ represents hydrogen atom, halogen atom (F, Cl, Br or I), OR₁₀, or SR₁₀, wherein R₁₀ represents hydrogen atom or an acyl group derived from a linear, branched or cyclic aliphatic acid or an aromatic acid. One or more of the same or different R₆ may substitute the aromatic ring.

Y₁ and Y₂ are the protecting group of R₄ and R₅, and represent

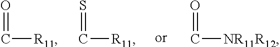

wherein R₁₁ and R₁₂ represent hydrogen atom, a linear, branched or cyclic C₁₋₁₀ alkyl group which may be substituted with aromatic groups.

In one preferred embodiment, the present invention provides a novel use of (S)-isoforms of isoproterenol and its analogs, for preventing diabetic cataracts and related diseases. These compounds satisfy several criteria important for this application. First of all, the anti-glycation activity of the (S)-isoform of isoproterenol and its analogs is high. Table 1 shows the IC₅₀ values of (S)-norepinephrine (IC₅₀=40.3±4.7 μM) and (S)-isoproterenol (IC₅₀=16.8±0.8 μM) that are essentially equivalent to those of (R)-norepinephrine (IC₅₀=39.6±3.4 μM) and (R)-isoproterenol (IC₅₀=18.1±1.1 μM), respectively.

TABLE 1
The values of the IC₅₀ and the values for the Iₘₐₓ of epinephrines

| Compound | Salt form | IC₅₀ (μM) | Iₘₐₓ (%) |
|---|---|---|---|
| (R)-Epinephrine | bitartrate | 15.5 ± 1.7 | 83 |
| (R,S)-Epinephrine | hydrochloride | 17.0 ± 1.1 | 82 |
| (R)-Isoproterenol | hydrochloride | 18.1 ± 1.1 | 86 |
| (S)-Isoproterenol | bitartrate | 16.8 ± 0.8 | 81 |
| (R)-Norepinephrine | bitartrate | 39.6 ± 3.4 | 92 |
| (S)-Norepinephrine | bitartrate | 40.3 ± 4.7 | 93 |

On this basis, it is reasonable to expect IC₅₀ values of (S)-epinephrine and its analogs as remaining in this range. Secondly, the adrenergic activity of the (R)-isoform, resulting in reducing the intra-ocular pressure, is insignificant for the (S)-isoform. The adrenergic activity of the (S)-isoform of epinephrine and its analogs is at least two orders of magnitude lower that that of the corresponding (R)-isoform (Patil et al., 1974). In particular, topical administration of up to 20% (S)-isoproterenol hydrochloride did not show any indication to lower intra-ocular pressure in the human eye (Kass et al., 1976).

The (S)-isoform of isoproterenol and its analogs are known to be safe for ocular administration. Various commercial preparations for the treatment of glaucoma contain (R,S)-epinephrine dipivalate (dipivefrin), which is a prodrug hydrolyzed to (R,S)-epinephrine after application to the eye. The liberated epinephrine contains equal amounts of the (S)- and (R)-isoform of epinephrine, of which only the adrenergically active (R)-isoform is relevant to the treatment of glaucoma. The (S)-isoform is inactive for this application, but its presence was proven to be safe. As preparations according to one preferred embodiment of the present invention contain only the (S)-isoform of isoproterenol and its analogs, they are also safe for ocular applications.

Isoproterenol is known to have the duration long enough for a reasonable frequency of administration, such as a twice-a-day administration, e.g., Bonomi (1964) instilled 2.47% (R,S)-isoproterenol to normal human eyes and observed a 20% reduction in ocular tension, lasting at least 12 h.

Once (S)-isoproterenol gets into blood circulation system, it is metabolized to 3-methyl-(S)-isoproterenol and its plasma half-life is in the range from 3.0 to 4.1 min (Conway et al., 1968), minimizing the possibility of any systemic adverse effects of (S)-isoproterenol.

For the use according to the invention, compounds of formula (I), in particular (S)-isoproterenol and its analogs, can be used in the form of their physiologically tolerated salts, physiologically functional derivatives, or prodrugs of formula (II). Preferred prodrugs or physiologically functional derivatives of compounds of formula (I) are those comprising at least one acyl group derived from a linear, branched or cyclic aliphatic acid or an aromatic acid, wherein the acyl group acylates at least one of X, R₃, R₄, R₅, or R₆. Pivaloyl (trimethylacetyl) acyl group is particularly preferred.

Compositions for the ocular treatment according to the present invention may contain one or more compounds of formula (I) and of formula (II), their physiologically tolerated salts, or physiologically functional derivatives. These compositions may be formulated in any dosage form suitable for topical ophthalmic delivery, such as solutions, suspensions, or emulsions. Of those, aqueous ophthalmic solutions are preferred. Other than the active ingredient(s), the compositions may further contain customary ophthalmic additives and excipients, such as antimicrobial preservative(s), viscosity-increasing agent(s) in order to increase the retention of the drugs and prodrugs, buffering agent(s), osmolarity-adjusting agent(s), surfactant(s), and antioxidant(s), if required or appropriate.

The formulated solution can be used as eye drop or applied by other methods such as soaking into soft contact lenses, which may reduce the effective concentration of the drugs or prodrugs with long duration (Bietti, et al., 1976).

### EXPERIMENTAL

### Materials and Methods

Chemicals: (S)-isoproterenol bitartrate, D-mannitol, benzalkonium chloride, pivaloyl chloride (trimethylacetyl chloride), and disodium sulfate were purchased from Aldrich (Oakville, Ontario, Canada). Chlorobutanol, aminocaproic acid, sodium perchlorate, hexadecylpyridinium chloride, [Glu¹]-fibrinopeptide B, and povidone (K30) were obtained from Sigma (Oakville, Ontario, Canada). Acetone, methylene chloride, glacial acetic acid, disodium carbonate, sodium chloride and NaOH were from EMD Science (Gibbstown, N.J., USA). Disodium edetate, trifluoroacetic acid (TFA), and water were purchased from J. T. Baker (Phillipsburg, N.J., USA). 1.0 M HCl was obtained from VWR (Montreal, Quebec, Canada). Water for mass spectrometry was purchased from Anachemia (Lachine, QC, Canada). Formic acid was purchased from Riedel de Haën (Oakville, Ontario, Canada). Acetonitrile was from Fisher Scientific (Nepean, Ontario, Canada). All the chemicals were used without further purification.

Hexadecylpyridinium acetate was prepared from hexadecylpyridinium chloride. Hexadecylpyridinium chloride was dissolved in methanol, and acetic acid and sodium acetate were added. After evaporating the solvent, the residue was dissolved in methylene chloride. Hexadecylpyridinium acetate was soluble in methylene chloride, whereas sodium chloride was precipitated and removed by filtration. The solvent was evaporated and the absence of chloride ion was confirmed as no precipitate was formed when silver nitrate solution was added to the product.

The purity of (S)-isoproterenol dipivalate hydrochloride was examined by Waters analytical HPLC system (600-MS controller, 600E pump, 717 autosampler, 996 photodiode array detector). The optical purity of (S)-isoproterenol bitartrate and (S)-isoproterenol dipivalate hydrochloride was examined by using another Waters HPLC system (600 controller, 600E pump, 717 autosampler, an d2996 photodiode array detector). High performance displacement chromatography (HPDC) was also carried out by using the latter system. NMR spectra were measured by Bruker Avance 500 MHz NMR. High-resolution mass spectra were measured by Micromass Waters Q-T of Ultima™ GLOBAL mass spectrometer (Mississauga, Ont, Canada) with NanoLockspray ([Glu¹]-fibrinopeptideB as a reference compound).

A rat model was used to study the effect of (S)-isoproterenol on diabetic cataract, that of streptozotocin-injection to induce diabetes by destroying the β-cells of the pancreas. This model has been widely used to study diabetes-related pathological events and their possible treatments (Dagher et al., 2004; Ben-nun et al., 2004; Chung et al., 2005). The rat model has an advantage of rapid formation of diabetic cataract such that initial cataract was observed as early as 8-9 weeks in the majority of the control diabetic rats. Two-month-old male Sprague-Dawley rats were purchased from Charles River, Canada. They were housed in the Biotechnology Research Institute (BRI)—animal facility. Housing and all experimental manipulations were approved by the BRI Animal Care Committee that functions under the guidelines of the Canadian Council of Animal Care.

(S)-isoproterenol dipivalate hydrochloride was synthesized from (S)-isoproterenol bitartrate. Pivaloyl chloride (trimethylacetyl chloride) (4.1 mmol, 500 mL) was added to a solution of (S)-isoproterenol bitartrate (1.0 mmol, 361.3 mg) in 50% 1M NaOH aq/acetone (5.5 ml, 5.5 mL). The mixture was allowed to react at room temperature for 1 h. The solution was acidified to pH 3-5 using 1.0 N HCl. After washing with n-hexane (Fisher; Nepean, Ontario, Canada), the solution was extracted with CH₂Cl₂. The organic layer was washed with 10% Na₂CO₃ aq solution, dried over anhydrous Na₂SO₄, and concentrated under reduced pressure. The residue was purified by high performance displacement chromatography (column; Shiseido Capcell PAK C18 AQ 5 μm; 250×4.6 mm; 4.0 mg/mL hexadecylpyridinium acetate 0.1% acetic acid in water, flow rate; 1.0 mL/min). The product was eluted out by a displacer, 4.0 mg/ml hexadecylpyridinium acetate 0.1% acetic acid in water. After salt exchange using 0.1 N HCl and lyophilization, (S)-isoproterenol dipivalate hydrochloride was obtained in 32±4% yield and 97.2±0.7% purity based on the quantitation of the impurities described below.

NMR (500 MHz, CD₃OD) δ 7.33 (dd, 1H, J=7.2, 1.9 Hz, Bz-6), 7.24 (d, 1H, J=1.9 Hz, Bz-2), 7.15 (d, 1H, J=7.2 Hz, Bz-5), 4.96 (dd, 1H, J=9.9, 3.1 Hz, CHOH—CH₂—NH₂), 3.40 (m, 1H, i-Pr(CH)), 3.18 (dd, 1H, J=12.3, 3.1 Hz, CHOH—CH₂—NH₂), 3.07 (dd, 1H, J=12.62, 9.9 Hz, CHOH—CH₂—NH₂), 1.32 (d, 6H, J=7.0 Hz, i-Pr(CH₃)), 1.30 (s, 9H, t-Bu), 1.29 (s, 9H, t-Bu).

High resolution electrospray ionization mass spectrometry of (S)-isoproterenol dipivalate, [MH]⁺_{calc.}=380.2437 and [MH]⁺_{obs.}=380.2426

The cytotoxicity of (S)-isoproterenol dipivalate was measured by using PC12 cells. PC12 cells (ATCC-CRL-1721) were grown in complete medium (RPMI 1640 medium supplemented with 10% heat-inactivated horse serum (Gibco), 5% calf serum (Hyclone) and 1× Penicillin/Streptomycin solution (Multicell)) and maintained at 37° C. in a humidified atmosphere containing 5% CO₂. PC12 cells were seeded onto rat-tail collagen coated 96-well plates at a density of 2×10⁴ cells/well and cultivated for one day. On the day of the experiment, dilutions of (S)-isoproterenol dipivalate ranging from 100 μM to 10 mM were prepared in complete medium. The medium was aspirated and the treatments were applied to the cells (in triplicates) for different incubation times (5 minutes to 2 hours). At the end of the incubation times, the medium was aspirated and 100 μL of a solution of MTT (3-(4,5-dimethylthiazol-2-yl)-2,5-diphenyltetrazolium; 0.2 mg/ml in complete medium) was added to the cells. After incubation at 37° C. for 2 hours, MTT was removed and the colored formazan was dissolved in dimethyl sulfoxide (DMSO) (½ hour at 37° C.). Reduction was measured by colorimetric detection (595 nm) of the blue insoluble formazan product. This assay provides an estimate of the number of functioning mitochondria present in the cells; i.e. the quantity of formazan product is directly proportional to the number of metabolically active cells in the culture. The viability of PC12 cells in each well was presented as percentage of control cells.

The cytotoxicity of (S)-isoproterenol dipivalate was measured by using human corneal epithelial cells (HCEC cells; Cascade Biologics). HCEC cells were grown in EpiLife medium supplemented with human corneal growth supplements (HCGS; Cascade Biologics). The cells were maintained at 37° C. in a humidified atmosphere containing 5% CO2 and the medium was changed every other day. HCEC cells were seeded onto 96-well plates at a density of approximately 10³ cells/well and cultivated for one day. On the day of the experiment, dilutions of S-isoproterenol dipivalate ranging from 250 μM to 25 mM were prepared in EpiLife medium. The medium was aspirated and the treatments were applied to the cells (in triplicates) for different incubation times (5 minutes to 2 hours and 24 hours). At the end of the incubation times, the survival of the cells was visually examined under microscope.

The Maillard fluorescence-based assay was used to screen anti-glycation activity of approximately 1,300 drugs or drug candidates. The details of the experimental conditions are described by Yeboah et al. (2002). Briefly the assay involved incubation of bovine serum albumin (BSA) (0.075 mM) with D-ribose (50 mM) and an assay compound (0.47, 4.7 and 47 μg/mL). Solutions were incubated at 37° C. for 5 days. Positive control, i.e., 100% inhibition of the Maillard fluorescence formation (370 nm excitation wavelength and 440 nm emission wavelength), consisted of a solution with BSA only. Negative control, i.e., no inhibition of the Maillard fluorescence formation, consisted of BSA with D-ribose. The assay compounds that had strong fluorescence or showed fluorescence quenching of Maillard fluorescence were excluded from the assay.

Two impurities were detected in (S)-isoproterenol dipivalate hydrochloride by HPLC using Waters SymmetryShield™ column (50×4.6 mm; 3.5 μm; water-acetonitrile linear gradient (0-80% in 7 min); flow rate, 2.0 mL/min). Both water and acetonitrile contained 0.1% TFA. They were identified as (S)-isoproterenol monopivalate hydrochloride based on their high resolution MS, i.e., [MH]⁺_{obs.}=296.1861, whereas [MH]⁺_{calc.}=296.1862. They were quantitated as 0.35±0.19% and 0.48±0.22% based on their absorption at 264 nm. These two monopivalate impurities were slowly interconverted and could not be analyzed separately (Wall et al., 1992)

The optical isomers of (S)-isoproterenol bitartrate and (S)-isoproterenol dipivalate hydrochloride were separated by HPLC using Shiseido chiral CD-Ph column (250×4.6 mm; 5 μm; isocratic 60:40 of 0.5 M sodium perchlorate/water and acetonitrile; flow rate, 1.0 mL/min). The elution profile was monitored by the absorption at 223 nm for isoproterenol bitartrate and 264 nm for isoproterenol dipivalate hydrochloride. The optical impurities were quantitated by the absorbance at 223 nm for isoproterenol bitartrate and 264 nm for isoproterenol dipivalate hydrochloride and by using a curve fitting software TABLECurve2D (Systat). The impurities of (R)-isoproterenol bitartrate and (R)-isoproterenol dipivalate hydrochloride were estimated as 2.0±0.3% and 3.3±0.2%, respectively. Thus, the racemization induced during synthesis and purification was minimal, if it occurred.

Another advantage of adrenalines is the formulation, i.e., commercial eye drop dipivefrin is a prodrug of (R,S)-epinephrine. It is more lipophilic than epinephrine, is still water soluble, is stable in eye drop solution, releases epinephrine when it passes through cornea, and pivalic acid, cleaved form of the blocking group, has a wide margin of safety, even at large oral administration. Dipivefrin enhances the ocular absorption 17 time better than epinephrine, allowing one to reduce the amount of the dose and the potential side effects (Mandell & Stentz, 1978). The same formulation of prodrug was successfully applied to isoproterenol (Hussain & Truelove, 1975). Thus, the effect of the formulated (S)-isoproterenol dipivalate hydrochloride on cataract with rat model was examined.

Active ingredient in the eye drop is 0.10% (w/v) (S)-isoproterenol dipivalate hydrochloride, and inactive ingredients are 1.84% (w/v) D-mannitol, 0.005% (w/v) disodium edetate, 0.10% (w/v) chlorobutanol, 0.16% (w/v) □-aminocaproic acid, 0.5% (w/v) sodium chloride, 0.003% (w/v) benzalkonium chloride, and 0.20% (w/v) povidone. The pH of the eye drop was adjusted to 5.5 with 1N—HCl. The control eye drop has the same inactive ingredients, but lacks the active ingredient. The eye drop was freshly prepared every month and was stored at 4° C. No degradation of the active and inactive gradients was detected based on their HPLC profiles after one month of storage at 4° C. The size of each eye drop was 50 μL.

Diabetes was induced in male Sprague-Dawley rats weighing approximately 200 to 250 g by a single intraperitoneal injection of the beta-cell toxin, Streptozotocin (STZ) (Sigma, St. Louis, Mich.), at a dose of 60 mg/kg body weight in 0.1M citrate buffer pH 4.5. Non-diabetic control rats received citrate buffer only.

One week following induction of diabetes, glucose levels were determined in the blood sampled from the tail vein using a blood glucose monitoring system (Ascensia ELITE Blood Glucose Meter, Bayer Inc, Toronto, ON, Canada). Since the limit of detection of the blood glucose meter was 33 mM, any value above that has been assigned a maximum value of 35 mM. Only animals with blood glucose levels higher than 15 mM were retained in the study. Animals were thus allocated into one of 4 groups: Group I (n˜20): Non-diabetic rats-receiving vehicle; Group II (n˜20): Non-diabetic rats-receiving eye drops containing (S)-isoproterenol dipivalate; Group III (n˜20): Diabetic rats-receiving vehicle; Group IV (n˜20): Diabetic rats-receiving eye drops containing (S)-isoproterenol dipivalate.

Eye drops or vehicle were administered twice a day, seven days a week, on the cornea of different groups of rats, with a minimum interval of 7 h between the two treatments. To promote weight gain and limit hyperglycemia, diabetic rats were injected sub-cutaneously with 2 IU ultralente insulin (Humulin, Eli Lilly, Toronto, ON, Canada) three times a week. Animal weights were monitored every week.

Cataract progression was monitored by visual examination, twice a week. A scoring system was devised to evaluate the severity of the cataract. A healthy eye was given a score of 0 (normal level); when a faint pinkish hue was discernable (level 1), cataract formation was in its earliest stage of being visually detected and this stage was given a score of 1. When a white film is clearly detectable (level 2), a score of 2 was assigned. When the film covers the entire eye, but the pupils are still visible (level 3), a score of 3 was assigned, and finally the cataract was considered most severe (score of 4) when the pupil was not detected due to the formation of the white film (level 4).

TABLE 2
Initiation and Progression of Diabetic Cataracts.

| | Time stayed in | | Time stayed in | | Time stayed in | | Time stayed in | | Time entered in | |
|---|---|---|---|---|---|---|---|---|---|---|
| | level 0 (week) | | level 1 (week) | | level 2 (week) | | level 3 (week) | | level 4 (week) | |
| Diabetic rats | Right eye | Left eye | Right eye | Left eye | Right eye | Left eye | Right eye | Left eye | Right eye | Left eye |
| Rat with | | | | | | | | | | |
| vehicle (control) | | | | | | | | | | |
| Rat 202 | 8 | 8 | 2 | 2 | 2.5 | 2.5 | 6 | 5 | 19.5 | 18.5 |
| Rat 212 | 8 | 10 | 1 | 1 | 2 | 1.5 | 2.5 | 1 | 14.5 | 14.5 |
| Rat 221 | 8 | 8 | 1 | 1 | 1 | 1 | 1 | 1.5 | 12 | 12.5 |
| Rat 232 | 8 | 9 | 1 | 2 | 2 | 2 | 10 | 0.5 | 22 | 14.5 |
| Rat 241 | 9 | 10 | 2 | 1 | 1 | 1.5 | 2.5 | 3 | 15.5 | 16.5 |
| Rat 251 | 8 | 9 | 1 | 1 | 3 | 3 | 6 | 5 | 19 | 19 |
| Rat 252 | 8 | 8 | 0 | 1 | 4 | 3 | 6 | 6 | 19 | 19 |
| Rat 261 | 8 | 8 | 0 | 0 | 4.5 | 2 | 4 | 6.5 | 17.5 | 17.5 |
| Rat 262 | 11.5 | 12 | 0.5 | 1 | 3.5 | 2.5 | NA | NA | □24 | □24 |
| Rat 271 | 7 | 7 | 2 | 2 | 2 | 2 | NA | NA | □24 | □24 |
| Rat 272 | 22 | >30 | 1 | NA^{a} | NA | NA | NA | NA | □24 | □30 |
| Rat 282 | 7 | 7 | 3 | 2 | 8 | 2 | NA | 4.5 | □24 | 16.5 |
| Rat 292 | 14 | 11.5 | 0.5 | 0.5 | 1 | 2.5 | 2.5 | 3.5 | 19 | 19 |
| Av ± STD | >10.2 ± 5.1 | | 1.18 ± 0.76 | | 2.5 ± 1.5 | | 4.1 ± 2.4 | | □19.2 ± 4.3 | |
| Rat with drug | | | | | | | | | | |
| (experimental) | | | | | | | | | | |
| Rat 311 | 8 | 8 | 1 | 1 | 2 | 2 | 2.5 | 2.5 | 14.5 | 14.5 |
| Rat 312 | 8 | 8 | 1 | 1 | 2 | 2 | 7.5 | 7.5 | 19.5 | 19.5 |
| Rat 321 | 8 | 9 | 1 | 2.5 | 2 | 1 | 7 | 5.5 | 19 | 19 |
| Rat 322 | 8 | 8 | 1 | 2 | 2 | 1.5 | 4 | 3 | 15.5 | 16 |
| Rat 331 | 13 | 14 | 0.5 | 0.5 | 0.5 | 0.5 | 3 | 3.5 | 19 | 18.5 |
| Rat 332 | 8 | 8 | 1 | 1 | 2 | 2 | 7 | 7 | 19 | 19 |
| Rat 341 | 18.5 | 18 | 2.5 | 3 | 2 | 2 | NA | NA | □24 | □24 |
| Rat 342 | 8 | 8 | 2 | 2 | 1.5 | 1.5 | 8 | 8 | 20.5 | 20.5 |
| Rat 351 | 17 | >30 | 0.5 | NA | 0.5 | NA | NA | NA | □24 | □30 |
| Rat 361 | >30 | >30 | NA | NA | NA | NA | NA | NA | □30 | □30 |
| Rat 362 | 18 | >23 | 0.5 | NA | 1 | NA | NA | NA | □24 | □30 |
| Rat 372 | 8 | 8 | 1 | 1 | 2 | 2 | 4 | 4 | 8 | 8 |
| Rat 381 | 14 | 9 | 0.5 | 3 | 5 | 2.5 | NA | NA | NA | NA |
| Rat 382 | >30 | >30 | NA | NA | NA | NA | NA | NA | □30 | □30 |
| Rat 391 | 11.5 | 9 | 0.5 | 2 | 2.5 | 5 | 6.5 | 5 | 22 | 22 |
| Rat 392 | 12 | 12 | 0 | 0.5 | 1 | 0.5 | 6.5 | 6.5 | 20.5 | 20.5 |
| Rat 401 | 18.5 | >30 | 1 | NA | 2.5 | NA | NA | NA | □24 | □30 |

| | | | | | |
|---|---|---|---|---|---|
| Av ± STD | >15.0 ± 8.3 | 1.24 ± 0.82 | 1.88 ± 1.09 | 5.4 ± 1.9 | □22.1 ± 4.9 |

^{d}NA: the cataract at this level is not developed.

### REFERENCES

Ben-nun, J., V. A. Alder, and I. J. Constable. 2004. Retinal microvascular patency in the diabetic rat. Int Ophthalmol. 25:187-192.
Bonomi, L. 1964. On the action of aleudrin on the normal human eye. Boll. Ocul. 43:412-420.
Chen, A. S., T. Taguchi, M. Sugiura, Y. Wakasugi, A. Kamei, M. W. Wang, and I. Miwa. 2004. Pyridoxal-aminoguanidine adduct is more effective than aminoguanidine in preventing neuropathy and cataract in diabetic rats. Horm. Metab. Res. 36:183-187.
Chung, H. K., S. M. Choi, B. O. Ahn, H. H. Kwak, J. H. Kim, and W. B. Kim. 2005. Efficacy of troxerutin on streptozotocin-induced rat model in the early stage of diabetic retinopathy. Arzneimittelforschung. 55:573-580.
Conway, W. D., H. Minatorya, A. M. Lnds, and J. M. Shekosky. 1968. Absorption and elimination profile of isoproterenol III. The metabolic fate of dl-isoproterenol-7-3H in the dog. J. Pharm. Sci. 1968, 57:1135-1141.
Dagher, Z., Y. S. Park, V. Asnaghi, T. Hoehn, and C. Gerhardinger. 2004. Studies of rat and human retinas predict a role for the polyol pathway in human diabetic retinopathy. Diabetes 53:2404-2411.
Hegde, K. R., M. G. Henein, and S. D. Varma. 2003. Establishment of the Mouse as a Model Animal for the Study of Diabetic Cataracts. Ophthalmic Res. 35:12-18
Hussain, A., and J. E. Truelove, 1975. Ester of 3,4-dihydroxy-alpha (isopropylamino) methyl benzyl alcohol, composition and anti-asthma use thereof. U.S. Pat. No. 3,868,461 (to INTERx Research Corp., Lawrence, Kans.).
Javinena, T., and K. Jarvinenb 1996. Prodrugs for improved ocular drug delivery. Adv. Drug Deliv. Rev. 19:203-224.
Kass M. A., T. W. Reid, A. H. Neufeld, L. P. Bausher, and M. L. Sears. 1976. The effect of d-isoproterenol on intraocular pressure of the rabbit, monkey, and man. Invest. Ophthalmol. 15:113-118.
Kyselova, Z., M. Stefek, and V. Bauer. 2004. Pharmacological prevention of diabetic cataract. Journal of Diabetes and its Complications 18:129-140.
Mandell, A. I., and F. Stentz. 1978. Dipivalyl epinephrine: A new pro-drug in the treatment of glaucoma. Ophthalmology 85:268-275.
Packer, L., K. Kraemer, and G. Rimbach, 2001, Molecular aspects of lipoic acid in the prevention of diabetes complications. Nutrition 17:888-895.
Patil, P. N., D. D. Miller, and U. Trendelenburg. 1974, Molecular geometry and adrenergic drug activity. Pharmacol. Rev. 26:323-392.
Rowland, J. M. and D. E. Potter. 1981, Steric structure activity relationships of various adrenergic agonists: ocular and systemic effects. Curr. Eye Res. 1:25-35.
Stitt, A. W. 2001. Advanced glycation: an important pathological event in diabetic and age related disease. Br. J. Opthalmol. 85:746-753.
Wall, G. M., j. C. Kenny, T. Y. Fan, C. Schafer, M. A. Ready, J. K. Baker, P. Ritter, and B. S. Scott. 1992. Analysis of 3- and 4-monopivaloylepinephrine, degradation products in dipivefrin hydrochloride drug substance and ophthalmic formulations. J. Pharm. Biomed. Anal. 10:465-471.
Wang, B. C., D. D. Bloxham, and K. L. Goetz. 1977, Effect of dipivalyl derivatives of catecholamines on cardiovascular function in the conscious dog. J. Pharmacol. Exp. Ther. 203:442-448.
Yan, H., and J. J. Harding. 2005. Carnosine protects against the inactivation of esterase induced by glycation and a steroid. Biochimica et Biophysica Acta 1741:120-126.
Yeboah, F., S. J. Cho, J. Lertvorachon, T. Kiyota, T. Popek, and Y. Konishi. 2002. Identification of novel antiglycants for the treatment of diabetes complications. PCT patent application (PCT/CA02/01552).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A method of preventing or delaying onset of diabetic cataracts in a subject, the method comprising topically administering a pharmaceutically effective amount of a compound of formula (I) 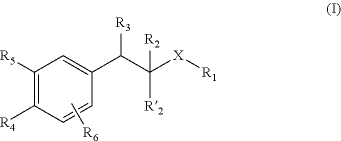
wherein
X represents NR_{S}, wherein R₇ represents hydrogen atom or an acyl group derived from a linear, branched or cyclic C₁₋₁₀ aliphatic acid or from an aromatic acid having a C₆₋₁₀ aromatic group,
R₁ represents hydrogen atom, NH₂, or a linear, branched or cyclic C₁₋₁₀ alkyl which may be substituted with a C₆₋₁₀ aromatic group,
R₂ represents hydrogen atom, a linear, branched or cyclic C₁₋₁₀ alkyl, or COOH group,
R′₂ represents hydrogen atom or a linear, branched or cyclic C₁₋₁₀ alkyl group,
R₃ represents hydrogen atom, ═O, OR₈, SR₈, or NR₈R₉, wherein R₈ and R₉ represent hydrogen atom, a linear, branched or cyclic C₁₋₁₀ alkyl, or an acyl group derived from a linear or branched C₁₋₁₀ aliphatic acid or from an aromatic acid having a C₆₋₁₀ aromatic group, provided that R₈ and R₉ are not both an acyl group,
R₄ and R₅ each independently represents OH, NH₂, or SH,
R₆ represents hydrogen, F, Cl, Br, I, OR₁₀, or SR₁₀, wherein R₁₀ represents hydrogen or an acyl group derived from a linear or branched C₁₋₁₀ aliphatic acid or from an aromatic acid having a C₆₋₁₀ aromatic group, R₆ may be present more than once and each R₆ may be the same or different,
a physiologically tolerated salt, prodrug or mixture thereof, to an eye of the subject in need of preventing or delaying onset of diabetic cataracts.

59. The method according to claim 58, wherein X is NH.

60. The method according to claim 59, wherein R₁ is —CH(CH₃)₂.

61. The method according to claim 60, wherein R₂ is H.

62. The method according to claim 61, wherein R′₂ is H.

63. The method according to claim 62, wherein R₃ is OH.

64. The method according to claim 63, wherein the compound has S-configuration, in which contamination of R-isomer is less than 3% w/w sufficient to reduce undesired adrenergic effects and other side effects of the R-isomer.

65. The method according to claim 64, wherein R₆ is H and R₄ and R₅ are both OH.

66. The method according to claim 58, wherein the compound is a prodrug.

67. The method according to claim 66, wherein the prodrug comprises at least one acyl group derived from a linear, branched or C₁₋₁₀ cyclic aliphatic acid or from an aromatic acid having a C₆₋₁₀ aromatic group.

68. The method according to claim 67, wherein the acyl group acylates at least one of X, R₃, R₄, R₅ or R₆.

69. The method according to claim 68, wherein the acyl group is pivaloyl.

70. The method according to claim 69, wherein X is NH, R₁ is isopropyl, R₃ is hydroxy, R₂, R′₂ and R₆ are hydrogen, R₄ and R₅ are pivaloylated hydroxy groups, and wherein the compound has S-configuration.

71. The method according to claim 58, wherein the prodrug is a compound of formula (II) 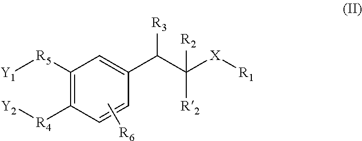
wherein:
X represents NR₇, wherein R₇ represents hydrogen atom or an acyl group derived from a linear, branched or C₁₋₁₀ cyclic aliphatic acid or from an aromatic acid having a C₆₋₁₀ aromatic group,
R₁ represents hydrogen atom, NH₂, or a linear, branched or cyclic C₁₋₁₀ alkyl which may be substituted with a C₈₋₁₀ aromatic group,
R₂ represents hydrogen atom, a linear, branched or cyclic C₁₋₁₀ alkyl, or COOH group,
R′₂ represents hydrogen atom or a linear, branched or cyclic C₁₋₁₀ alkyl group,
R₃ represents hydrogen atom, ═O, OR₈, SR₈, or NR₈R₉, wherein R₈ and R₉ represent hydrogen atom, a linear, branched or cyclic C₁₋₁₀ alkyl, or an acyl group derived from a linear, branched or C₁₋₁₀ cyclic aliphatic acid or from an aromatic acid having a C₆₋₁₀ aromatic group, provided that R₈ and R₉ are not both an acyl group,
R₄ and R₅ each independently represents —O—, —NH— or —S—,
R₆ represents hydrogen atom, F, Cl, Br, I, OR₁₀, or SR₁₀, wherein R₁₀ represents hydrogen atom or an acyl group derived from a linear, branched or C₁₋₁₀ cyclic aliphatic acid or from an aromatic acid having a C₆₋₁₀ aromatic group, R₆ may be present more than once and each R₆ may be the same or different,
Y₁ and Y₂ are protecting groups of R₅ and R₄ respectively, and represent 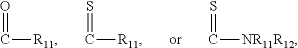
wherein R₁₁ and R₁₂ represent hydrogen atom, a linear, branched or cyclic C₁₋₁₀ alkyl group which may be substituted with one or more C₆₋₁₀ aromatic groups,
a physiologically tolerated salt, or mixture thereof.

72. The method according to claim 71, wherein X is NH.

73. The method according to claim 72, wherein R₁ is —CH(CH₃)₂.

74. The method according to claim 73, wherein R₂ is H.

75. The method according to claim 74, wherein R′₂ is H.

76. The method according to claim 75, wherein R₃ is OH.

77. The method according to claim 76, wherein the compound has S-configuration, in which contamination of R-isomer is less than 3% w/w sufficient to reduce undesired adrenergic effects and other side effects of the R-isomer.

78. The method according to claim 77, wherein R₆ is H and R₄ and R₅ are both —O—.

79. The method according to claim 78, wherein Y₁ and Y₂ are both pivaloyl.

80. The method according to claim 58, wherein the compound is α-(1-methyl-3-phenyl-propylamino)-3,4-dihydroxyacetophenone, 3,4-dihydroxy-1-[α-(1-methyl-3-phenyl-propylamino)-β-hydroxyethyl]benzene, 3,4-dihydroxy-1-[α-isopropylamino-β-methoxy)ethyl]benzene, 3,4-dihydroxy-1-[(α-methylamino-β-methoxy)ethyl]benzene, isoetharine, (S)-isoproterenol, S(−)-carbidopa or corbadrine.

81. The method according to claim 58, wherein the compound is (S)-isoproterenol, (S)-isoproterenol dipivalate, a physiologically tolerated salt or a mixture thereof.

82. The method according to claim 58, wherein the compound is formulated as a topical ophthalmic solution.
